(19) United States
(12) Patent Application Publication
(10) Pub. No.: US 2022/0251657 A1
(43) Pub. Date: Aug. 11, 2022

(54) GENOMIC DIAGNOSTIC METHOD AND KIT TO DIAGNOSE EARLY ESOPHAGEAL CANCER AND BARRETTS ESOPHAGUS

(71) Applicant: Board of Regents, The University of Texas System, Austin, TX (US)

(72) Inventor: Ikenna Okereke, Galveston, TX (US)

(21) Appl. No.: 17/582,510

(22) Filed: Jan. 24, 2022

Related U.S. Application Data
(60) Provisional application No. 63/143,515, filed on Jan. 29, 2021.

(51) Int. Cl. C12Q 1/6883; G01N 33/50; C12Q 1/689; C12Q 1/6886

(52) U.S. Cl. CPC C12Q 1/6883; G01N 33/5044; C12Q 2600/158; C12Q 1/6886; C12Q 2600/112; C12Q 1/689

(57) ABSTRACT

The present invention includes an ex vivo model of an esophagus comprising: a well comprising a cellular growth media; a substrate comprising an apical surface and a nutrient-rich basal surface on which an air-liquid interface is formed at the media, wherein an epithelial layer is grown on the apical surface; and wherein a squamous epithelial layer of esophageal cells or cell lines is grown on the apical surface of the ex vivo system.

### CROSS-REFERENCE TO RELATED APPLICATIONS

This application claims priority to U.S. Provisional Application Ser. No. 63/143,515, filed Jan. 29, 2021 entitled “Genomic Diagnostic Method and Kit to Diagnose Early Esophageal Cancer and Barretts Esophagus”, which is hereby incorporated by reference in its entirety.

### INCORPORATION-BY-REFERENCE OF MATERIALS FILED ON COMPACT DISC

Not applicable.

### TECHNICAL FIELD OF THE INVENTION

The present invention relates in general to the field of detection of microorganisms in the esophagus, and more particularly, to novel methods for determining the presence of Barrett's esophagus and cancer.

### STATEMENT OF FEDERALLY FUNDED RESEARCH

Not applicable.

### BACKGROUND OF THE INVENTION

Without limiting the scope of the invention, its background is described in connection with esophageal disease and cancer.

Despite advancements in surgical management and chemoradiation protocols, esophageal adenocarcinoma (EAC) remains one of the most lethal gastrointestinal malignancies with an overall 5 year survival rate of 17% and a proclivity for detection at an advanced stage [1-2]. Intestinal metaplasia of the distal esophagus, known as Barrett's esophagus (BE), can occur in patients with chronic inflammation of the distal esophagus and is a critical risk factor for development of EAC. Patients with BE are recommended to undergo increased surveillance screening with endoscopic examination and biopsy of the esophagus [3]. BE is a crude risk factor, however, and only 1 in 860 patients with BE ultimately develop EAC [4].

Over the last several years, multiple studies have identified differences in the microbial community within the esophagus in people with BE compared to people without BE [5-6]. The normal esophagus is characterized by colonization with a high prevalence of Firmicutes phyla, of which Streptococcus is a member, while the esophagus of people with BE has a much higher percentage of gram negative anaerobes [7]. The different microbial communities suggest two possibilities. Either the altered microbial community changes the mucosa of the esophagus or the altered intraluminal environment of the esophagus in patients with BE changes the microbial community. Yang and colleagues have proposed that gram negative organisms play an important role in esophageal carcinogenesis, due to the release of lipopolysaccharides (LPS) which activate the innate immune system and result in chronic inflammation [8]. Furthermore, LPS are implicated in the activation of the pro-inflammatory NF-KB pathway and the exacerbation of reflux through the inhibition of gastric emptying [9-10].

Although these previous studies have outlined the phenomenological differences between patients with and without esophageal disease, there has been minimal research determining the mechanism by which these changes in the microbial community occur. The microbiome has been increasingly associated with different disease processes, but its role in esophagus is largely unknown. What is needed is an understanding of the associations of the esophageal microbiota with Barrett's esophagus and methods for detecting and treating the same.

### SUMMARY OF THE INVENTION

In one embodiment, the present invention includes a method of detecting and treating a patient suspected of having Barrett's esophagus comprising: obtaining a biological sample from an esophagus of the patient; determining a transcriptome in the biological sample by detecting the presence of one or more genes selected from one or more of the genes in Table 4; wherein if the patient has a transcriptome indicative of an increased risk of esophageal cancer treating the patient with at least one of: removing at least part of the esophagus, esophagectomy, probiotic therapy, chemically targeting elimination of bacteria indicative of an increased risk of esophageal cancer or increasing the frequency of endoscopic surveillance, or chemotherapy. In one aspect, the genes are selected from 1, 2, 3, 4, 5, 6, 7, 8, 9, 10, 11, 12, 13, 14, 15 or more of the genes selected from FosB, Early growth response protein 1 (EGR1), Early growth response protein 3 (EGR3), Nuclear receptor subfamily 4 group A member 1 (NR4A1), Cyclic AMP-dependent factor ATF-3 (ATF3), Hepatocyte nuclear factor 4-alpha (HNF4A), Ankyrin repeat and SAM domain-containing protein 4B (ANKS4B), Galectin-4 (LGALS4), Apoptosis facilitator Bcl-2-like protein 14, Cyclin-dependent kinase inhibitor 2A (CDKN2A), Matrix metalloproteinase 7 (MMP7), E3 ubiquitin-protein ligase Mdm2 (MDM2), Regenerating islet-derived protein (REG), Calpain 8 (CAPN8), Defensin beta 103A (DEFB103A), Rho GTPase Activating Protein 26 (ARHGAP26), Cell division cycle 25B (CDC25B), and G protein subunit beta-2 (GNB2). In another method, the method further comprises determining a biome in the biological sample wherein an absence of Streptococcus salivarius, Actinomyces, Prevotella, or Dialister is indicative of an increased risk of esophageal cancer. In another method, the method further comprises determining a biome in the biological sample wherein an absence of Corynebacterium, Dialister, Gemella, Haemophilus, Leptotrichia, Neisseria, Prevotella, Rothia, Streptococcus, Veillonella is indicative of worsened severity of Barrett's esophagus and an increased risk of esophageal cancer. In another method, the method further comprises stratifying patients based on the presence of bacteria in the uvula, proximal esophagus and distal esophagus. In another aspect, the biological sample is an esophageal swab and mucosal biopsies were obtained from the uvula, proximal esophagus and distal esophagus. In another aspect, the expression of 2, 3, 4, 5, 6, 7, 8, 9, 10, 11 or 12 genes is indicative of Barrett's esophagus. In another aspect, the genes are on an array. In another aspect, the biological sample is obtained from the distal esophagus and a length of a Barrett's column correlated with a level of Barrett's esophagus leading to esophageal cancer. In another aspect, the biological sample is a breath sample, a saliva sample, or a biopsy of the esophagus. In another aspect, the presence of Barrett's esophagus is determined without use of age, gender or presence of hiatal hernia as a factor or factors.

In another embodiment, the present invention includes an ex vivo system of an esophagus comprising: a well comprising a cellular growth media; a substrate comprising an apical surface and a nutrient-rich basal surface on which an air-liquid interface is formed at the media, wherein an epithelial layer is grown on the apical surface; and wherein a squamous epithelial layer of esophageal cells or cell lines is grown on the apical surface of the ex vivo system. In another aspect, the model further comprises a microbiome harvested from one or more patients with Barrett's esophagus and putting in contact with the esophageal cells or cell lines. In another aspect, the model further comprises one or more Barrett's esophageal cell lines. In another aspect, a microbiome is inoculated onto a sterile apical surface of each esophageal cell line. In another aspect, a microbiome is be inoculated onto a sterile apical surface a Barrett's esophageal cell line.

In another embodiment, the present invention includes a kit for an ex vivo system of an esophagus comprising: a well comprising a cellular growth media; a substrate comprising an apical surface and a nutrient-rich basal surface on which an air-liquid interface is formed at the media, wherein an epithelial layer is grown on the apical surface adapted for the growth of a squamous epithelial layer of esophageal cells or cell lines is grown on the apical surface of the ex vivo system; and instructions for the growth for a squamous epithelial layer of esophageal cells or cell lines.

In another embodiment, the present invention includes a method of testing one or more compounds, microbiomes, or combinations there for efficacy in treating esophageal diseases in an ex vivo model of an esophagus comprising: providing a well comprising a cellular growth media; placing a substrate comprising an apical surface and a nutrient-rich basal surface on which an air-liquid interface is formed at the media, wherein an epithelial layer is grown on the apical surface; growing squamous epithelial layer of esophageal cells or cell lines is grown on the apical surface of the ex vivo system; contacting the squamous epithelial layer of esophageal cells or cell lines with the one or more compounds, microbiomes, or combinations; and detecting a change in the squamous epithelial layer of esophageal cells or cell lines.

In another embodiment, the present invention includes a method of determining an extent of disease progression in a patient suspected of having Barrett's esophagus comprising: obtaining a biological sample from one or more locations of an esophagus of the patient; determining or obtaining a transcriptome in the biological sample by detecting the presence of one or more genes selected from FosB, Early growth response protein 1 (EGR1), Early growth response protein 3 (EGR3), Nuclear receptor subfamily 4 group A member 1 (NR4A1), Cyclic AMP-dependent factor ATF-3 (ATF3), Hepatocyte nuclear factor 4-alpha (HNF4A), Ankyrin repeat and SAM domain-containing protein 4B (ANKS4B), Galectin-4 (LGALS4), Apoptosis facilitator Bcl-2-like protein 14, Cyclin-dependent kinase inhibitor 2A (CDKN2A), Matrix metalloproteinase 7 (MMP7), E3 ubiquitin-protein ligase Mdm2 (MDM2), Regenerating islet-derived protein (REG), Calpain 8 (CAPN8), Defensin beta 103A (DEFB103A), Rho GTPase Activating Protein 26 (ARHGAP26), Cell division cycle 25B (CDC25B), and G protein subunit beta-2 (GNB2); and matching the extent of Barrett's esophagus to disease progression by detecting the presence of the transcriptome at the one or more locations of the esophagus that correlate with different levels of Barrett's esophagus disease progression; and if the patient has a transcriptome is indicative or early Barrett's esophagus disease then increasing a frequency of Barrett's esophagus disease surveillance from every 3 months to every three years in three-month increments; or if the patient has a transcriptome indicative of advanced Barrett's esophagus treating the patient with at least one of: removing at least part of the esophagus, esophagectomy, probiotic therapy, chemotherapy, chemically targeting elimination of bacteria indicative of an increased risk of esophageal cancer or increased frequency of endoscopic surveillance. In another aspect, the method further comprises stratifying patients based on the presence of bacteria in the uvula, proximal esophagus and distal esophagus. In another aspect, the biological sample is an esophageal swab and mucosal biopsies were obtained from the uvula, proximal esophagus and distal esophagus. In another aspect, the expression of 2, 3, 4, 5, 6, 7, 8, 9, 10, 11 or 12 genes is indicative of Barrett's esophagus. In another aspect, the biological sample is obtained from the distal esophagus and a length of a Barrett's column correlated with a level of Barrett's esophagus leading to esophageal cancer. In another aspect, the biological sample is a breath sample, a saliva sample, a biopsy of the esophagus or a serum sample. In another aspect, the presence of Barrett's esophagus is determined without use of age, gender or presence of hiatal hernia as a factor or factors.

### BRIEF DESCRIPTION OF THE DRAWINGS

For a more complete understanding of the features and advantages of the present invention, reference is now made to the detailed description of the invention along with the accompanying figures and in which:

FIG. 1 shows a map of an EMB qPCR array.

FIG. 2 shows normal esophagus and adenocarcinoma immortalized cell lines following thawing from liquid nitrogen at 24 and 72 hours. Each image shows 4× magnification.

FIG. 3 shows two examples of devices for the ex vivo models of the present invention.

FIG. 4 shows the probability of detection of organisms.

FIG. 5 shows the probability of Detection of Organisms vs. Length of Barrett's Esophagus.

FIG. 6 shows cluster analysis data of 36 patients with and without Barrett's esophagus.

FIG. 7 shows an Ingenuity Pathway Analysis of processes related to Barrett's transcriptome.

FIG. 8 shows histology of normal EC mucosal layer successfully grown on air-interfaced plate.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

The present invention includes an ex vivo model for examining the mechanism by which host-microbe interactions occur within the esophagus. Using a monolayer of immortalized esophageal cells with an air-liquid interface, a normal microbiome and Barrett's microbiome can be transplanted directly onto the epithelial cell layer. This model can be used to examine the mechanism by which different microbial communities interact with the esophageal mucosa and the differences in transcription and metabolomics profiles. The model system of the present invention was used to perform to characterize the microbiome and transcriptome profile of the human esophagus. This human data demonstrates the basis by which the results from ex vivo experiments can be compared.

As used herein, the term “amplification” refers to methods that include, but are not limited to, polymerase chain reaction (PCR), ligation chain reaction (sometimes referred to as oligonucleotide ligase amplification OLA), cycling probe technology (CPT), strand displacement assay (SDA), transcription mediated amplification (TMA), nucleic acid sequence based amplification (NASBA), rolling circle amplification (RCA), and invasive cleavage technology. These methods require a primer nucleic acid (including nucleic acid analogs) that is hybridized to a target sequence to form a hybridization complex, and an enzyme is added that in some way modifies the primer to form a modified primer. For example, PCR generally requires two primers, dNTPs and a DNA polymerase; LCR requires two primers that adjacently hybridize to the target sequence and a ligase; CPT requires one cleavable primer and a cleaving enzyme; invasive cleavage requires two primers and a cleavage enzyme; etc. Thus, in general, a target nucleic acid is added to a reaction mixture that comprises the necessary amplification components, and a modified primer is formed.

These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

As used herein, the term “polynucleotide” refers to nucleic acid strands composed entirely of deoxyribonucleotides, entirely of ribonucleotides, or chimeric mixtures thereof. Polynucleotides may be comprised of internucleotide, nucleobase and sugar analogs. Unless denoted otherwise, whenever a polynucleotide sequence is represented, it will be understood that the nucleotides are in 5′ to 3′ orientation from left to right and that “A” denotes deoxyadenosine, “C” denotes deoxycytidine, “G” denotes deoxyguanosine, and “T” denotes thymidine.

As used herein, the term “polynucleotide template” refers to a region of a polynucleotide complementary to an oligomer, probe or primer polynucleotide. It is understood that a polynucleotide template will normally constitute a portion of a larger polynucleotide molecule, with the “template” merely referring to that portion of the polynucleotide molecule to which the oligomer, probe or primer of the present invention is complementary.

As used herein, the term “primer” refers to an oligonucleotide molecule that is complementary to a portion of a target sequence and, upon hybridization to the target sequence, has a free 3′-hydroxyl group available for polymerase-catalyzed covalent bonding with a 5′-triphosphate group of a deoxyribonucleoside triphosphate molecule, thereby initiating the enzymatic polymerization of nucleotides complementary to the template. Primers may include detectable labels for use in detecting the presence of the primer or primer extension products that include the primer.

As used herein, the term “probe” refers to a nucleobase oligomer that is capable of forming a duplex structure by complementary base pairing with a sequence of a target polynucleotide, and further where the duplex so formed is detected, visualized, measured and/or quantitated. In some embodiments, the probe is fixed to a solid support, such as in column, a chip or other array format. Probes may include detectable labels for use in detecting the presence of the probe.

As used herein, “target sequence” refers to a nucleic acid sequence on a single strand of nucleic acid. The target sequence may be a portion of a gene, a regulatory sequence, genomic DNA, cDNA, RNA including mRNA and rRNA, or others. As is outlined herein, the target sequence may be a target sequence from a sample, or a secondary target such as a product of an amplification reaction, a fragmentation reaction, and the like. A target sequence may be of any length. A target sequence often comprises a fragment of a target polynucleotide, and the length of that fragment may comprise some or all of the target polynucleotide from which it is derived.

As used herein, the term “template” refers to the region of the polynucleotide that constitutes the physical template for hybridization of another complementary polynucleotide. Templates may be genomic DNA, cDNA, PCR amplified DNA, or any other polynucleotide that serves as a pattern for the synthesis of a complementary polynucleotide.

The present invention may employ, unless otherwise indicated, conventional techniques of molecular biology, microbiology, recombinant DNA techniques, and oligonucleotide synthesis which are within the skill of the art. Such techniques are explained fully in the literature. Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)); Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.), the contents of all of which are incorporated herein by reference.

Kits. The present invention also includes kits that include reagents that can be used in practicing the methods disclosed herein. The kits can include any reagent or combination of reagent discussed herein or that would be understood to be required or beneficial in the practice of the disclosed methods, including certain arrays. For example, the kits could include primers to perform the amplification reactions discussed in certain embodiments of the methods, as well as the buffers and enzymes required to use the primers as intended. The kit can also include a standard or custom array for detecting amplification products. For example, disclosed is a kit comprising reagents for real-time PCR-type amplification reaction for detecting a Barrett's esophagus microbiome, comprising sense primers, antisense primers and a nondegenerate probe. For example the kit can detect a microbiome of Barrett's esophagus.

For enhanced diagnostics, the present invention used a qPCR array to diagnose individuals with Barrett's esophagus transcriptome changes and to monitor the success of therapeutic approaches. Another such array uses customized qPCR assays assembled into kitted systems for use in clinical microbiology facilities. The array can be used to identify optimal donated oral swab specimens from healthy donors to support development of therapeutic microbiome transplants. The therapeutic composition that include oral compositions, suppositories, enemas and/or serum samples that include supplementation with identified and cultured probiotic organisms reduced or lost in the microbiome of Barrett's esophagus patients.

In one example, the present invention detects 1 2, 3, 4, 5, 6, 7, 8, 9, 10, 11, 12, 13, 14, 15, 20, 25, 30, 40, 50, 60, 70, 75, 80, 90, 100, 150, 200, 250, 300, 350, 400, 500, 600, 700, 800, 900, or all the genes selected from: ISG15; PRXL2B; ESPN; ERRFI1; EFHD2; AGMAT; FBLIM1; PADI2; RCC2; AKR7L; PLA2G2A; LDLRAD2; HTR1D; CNR2; PAFAH2; SLC9A1; IFI6; DNAJC8; SESN2; RAB42; MEAF6; BMP8B; GUCA2B; GUCA2A; RIMKLA; SVBP; TMEM53; BEST4; TOE1; LURAP1; RAB3B; AL445685.3; USP1; GADD45A; PTGER3; TNNI3K; IFI44L; SAMD13; GBP3; BRDT; PALMD; SLC35A3; SLC30A7; GPSM2; KIAA1324; PSMA5; KCND3; RHOC; PTPN22; BCL2L15; AMPD1; REG4; NBPF20; NBPF10; FMO5; AC239811.1; MTMR11; SELENBP1; FLG; LCE3E; LCE3D; SPRR2F; PRR9; S100A7A; S100A7; AQP10; CD1E; IFI16; SLAMF7; ITLN1; FCGR2C; GPA33; F5; Clorf112; SELE; SUCO; AXDND1; LAMC2; RGS2; CRB1; LGR6; FMOD; ZBED6; GOLT1A; IL19; CR1; PROX1; RRP15; MARK1; CAPN8; TP53BP2; LEFTY1; RHOU; DISC1; GCSAML; LYPD8; CMPK2; ID2; HPCAL1; APOB; ATAD2B; NCOA1; KCNK3; TRIIVI54; RASGRP3; GALM; PLEKHH2; EPCAM; CNRIP1; ASPRV1; PAIP2B; ZNF638; DUSP11; ACTG2; LRRTM4; REG3G; REG3A; KIAA1211L; IL1R2; IL1RL1; FHL2; SULT1C2; ZC3H8; TFCP2L1; MYO7B; POTEI; PSMD14; GCA; NOSTRIN; METTLS; PDE1A; FSIP2; TFPI; C2orf88; CAVIN2; C2orf66; BOLL; PPIL3; CDK15; NRP2; SPAG16; TNS 1; AAMP; VIL1; DES; SPEGNB; GMPPA; ALPG; SNORC; UGT1A9; MLPH; HES6; GPR35; GHRL; TAMM41; TIMP4; RAF1; AC090004.1; BTD; KCNH8; LRRFIP2; VILL; VIPR1; SLC6A20; CCDC12; LAMB2; TCTA; AC104452.1; MST1R; SEMA3B; HYAL1; ACY1; GLYCTK; CHDH; APPL1; CEP97; CD47; CD96; HGD; STXBPSL; HCLS1; MCM2; RHO; NME9; SLC9A9; TM4SF4; TSC22D2; SUCNR1; PQLC2L; ARL14; GOLIM4; TNFSF10; FXR1; LAMP3; C3orf70; IGF2BP2; SST; TP63; IL1RAP; FAM43A; CEP19; MFSD10; WFS1; MRFAP1L1; FGFBP2; PROM1; LDB2; GBA3; OCIAD2; LRRC66; CENPC; STAP1; UGT2B11; SULT1B1; SULT1E1; SLC4A4; CXCL6; CXCL1; CXCL5; CXCL2; CXCL9; CXCL10; LIN54; ABCG2; ADH6; MTTP; DAPP1; SLC9B1; UGT8; KIAA1109; HSPA4L; ZNF330; HHIP; OTUD4; SMIM31; PALLD; NEK1; GALNT7; HPGD; DCTD; ING2; CASP3; SORBS2; LRRC14B; SLC9A3; SLC12A7; SLC6A19; OTULINL; RETREG1; SUB1; DNAJC21; IL7R; DAB2; OXCT1; FST; PPWD1; SREK1; CCDC125; IQGAP2; F2RL2; TBCA; MTX3; XRCC4; HAPLN1; ADGRV1; TRIM36; TICAM2; SEMA6A; SLC22A5; SMIM32; PFDN1; PCDHA1; RNF14; ARHGAP26; CDX1; NMUR2; GALNT10; FAXDC2; SGCD; MED7; RANBP17; AC139491.7; CDHR2; FGFR4; CLK4; ZNF354B; ZFP2; BTNL3; GMPR; NUP153; TPMT; MB OAT1; DCDCl₂; SLC17A4; HIST1H1D; TRIM31; TRIM15; DHX16; CDSN; LY6G5B; DXO; PPT2-EGFL8; GPSM3; MLN; RPS10-NUDT3; SPDEF; SCUBE3; CLPS; RAB44; KCNK16; AL096814.1; KLC4; MEP1A; MTO1; IMPG1; NT5E; SYNCRIP; SMPD2; LAMA4; ASF1A; NCOA7; C6orf58; BCLAF1; ARFGEF3; ECT2L; CCDC170; ZDHHC14; SOD2_1; SLC22A1; PHF10; ADAP1; LFNG; FSCN1; ICA1; TSPAN13; AGR2; IGF2BP3; HOXA9; HOXA10; HOXA13; CPVL; MRPL32; COA1; PURB; IGFBP3; EGFR; PHKG1; GUSB; ASL; DNAJC30; CCL26; FGL2; CD36; SEMA3C; CACNA2D1; SEMA3A; SEMA3D; KIAA1324L; CROT; SRI; CFAP69; GTPBP10; CDK6; GNGT1; ASB4; ARPC1B; ZCWPW1; GNB2; MUC3A; MUC17; MOGAT3; PLOD3; CLDN15; RASA4; POLR2J2; SLC26A3; PNPLA8; CFTR; CTTNBP2; FLNC; TMEM209; CPA2; CPA4; CPA1; KIAA1549; PRSS1; PRSS2; KEL; TMEM139; OR6B 1; PDIA4; A0C1; WDR86; NOM1; KBTBD11; DEFA5; DEFB4B; DEFB 103B; DEFB103A; DEFB4A; SOX7; GATA4; C8orf58; TRIM35; HMBOX1; DUSP4; BAG4; ZMAT4; PLAT; LY96; TPD52; PAG1; PMP2; FABP4; RUNX1T1; CDH17; AC012213.5; EMC2; MRPL13; ANXA13; FER1L6; SLC45A4; TSNARE1; GSDMD; PPP1R16A; ACER2; MOB3B; C9orf72; SPINK4; ANKRD18B; CA9; ZCCHC7; PRUNE2; KIF27; ROR2; BICD2; FBP1; AOPEP; FOXE1; HEMGN; SMC2; TAL2; EPB41L4B; C9orf43; TRAF1; NEK6; CEL; GPSM1; MAMDC4; ENTPD2; NRARP; UCN3; TUBAL3; PFKFB3; PROSER2; FAM171A1; MLLT10; OTUD1; BAMBI; SVIL; ZNF438; ZNF485; ZFAND4; MSMB; GPRIN2; ZNF488; PARG; A1CF; PRKG1; IPMK; PBLD; RUFY2; HKDC1; SLC29A3; DNAJC9; MYOZ1; SYNPO2L; KCNMA1; NUTM2B; IFIT2; IFIT3; IFIT1; PLCE1; CYP2C19; CYP2C9; HOGA1; SEMA4G; LDB1; NEURL1; SMC3; ADRA2A; VWA2; PNLIPRP1; PNLIPRP2; PRLHR; GRKS; CUZD1; GPR26; CLRN3; VENTX; PGGHG; IFITM3; CDHRS; CRACR2B; MUCSAC; SLC22A18AS; SLC22A18; PHLDA2; HBB; FAM160A2; CAVIN3; RIC3; STK33; SCUBE2; KCNJ11; ABCC8; PRMT3; GAS2; SVIP; SLC5A12; FIBIN; PAX6; TP53111; PTPRJ; OR8H1; SLC43A1; UBE2L6; SERPING1; MS4A2; MS4A1; PGA3; PGAS; FADS2; PLAAT3; PLCB3; BRMS1; LRFN4; C11orf86; AP003419.1; ACY3; TCIRG1; CHKA; C11orf24; ANAPC15; STARD10; PLEKHB1; KLHL35; PCF11; EED; RAB38; FUT4; MAML2; MMP1; POGLUT3; LAYN; TMPRSS5; HTR3B; APOA4; MPZL2; CD3E; CD3D; CD3 G; CCDC153; USP2; GRAMD1B; BARX2; ADAMTS15; SLC6A12; DYRK4; C1S; C1R; APOBEC1; PHC1; CLEC2B; KLRK1; EPS8; BHLHE41; PTHLH; PUS7L; IRAK4; PCED1B; DNAJC22; GPD1; SMIM41; KRT74; KRT76; KRT8; KRT18; PRR13; HOXC11; HOXC10; MUCL1; METTL7B; SLC39A5; RBMS2; MYO1A; R3HDM2; METTL1; HMGA2; MYRFL; TSPAN8; TRHDE; NAV3; CCDC59; EEA1; TMCC3; SNRPF; SLC41A2; C12orf75; TMEM263; SSH1; TCTN1; PPP1CC; SH2B3; OAS2; TBX3; SRRM4; HNF1A; TMEM120B; RHOF; HIP1R; KMT5A; RFLNA; CDX2; VWA8; SLC25A30; HTR2A; MED4; LPAR6; AL162377.3; ATP7B; DACH1; GPR183; COL4A2; TEP1; RNASE2; TMEM253; OR5AU1; RABGGTA; LTB4R2; CTSG; SSTR1; PPM1A; ZBTB1; PPP1R36; PLEK2; TMEM229B; FAM161B; ENTPD5; FLVCR2; DIO2; RIN3; CHGA; ITPK1; IFI27L2; AMN; CKB; INF2; GPR132; IGHD; NPAP1; AC124312.1; CHRNA7; ZNF770; PHGR1; DISP2; KNSTRN; PPP1R14D; CHAC1; LTK; PLA2G4E; TGM5; GLDN; WDR72; RAB27A; PIGBOS1; DNAAF4; GCNT3; TLN2; TPM1; DAPK2; RBPMS2; SLC51B; CILP; SMAD6; C15orf61; CALML4; SEMA7A; CYP1A1; SCAPER; TSPAN3; STARD5; GOLGA6L9; AC245033.1; TM6SF1; FANCI; ARPIN-AP3S2; MEF2A; PDIA2; MCRIP2; MSLN; TPSB2; TPSAB1; C16orf91; GFER; SLC9A3R2; PRSS33; IL32; MTRNR2L4; SMIM22; NAGPA; C16orf89; GRIN2A; TEKTS; CPPED1; BFAR; MYH11; GP2; ANKS4B; AC092338.1; ERN2; CLN3; SULT1A2; SULT1A1; NPIPB12; ZG16; MAZ; PRRT2; AC009133.6; KCTD13; AC093512.2; SLX1A; ZNF423; HEATR3; BRD7; CHD9; RPGRIP1L; MT2A; MT1E; MT1H; CCL22; MMP15; CA7; CES3; TPPP3; HSD11B2; PARD6A; NRN1L; PRMT7; SMPD3; COG8; CLEC18A; CLEC18C; AC010547.4; MARVELD3; HP; LDHD; ZFP1; CTRB2; CTRB1; TMEM170A; AC009163.2; CHSTS; HSD17B2; ATP2C2; ZFPM1; SPIRE2; TUBB3; PRDM7; DOC2B; RILP; ASPA; P2RX5-TAX1BP3; HASPIN; P2RX1; ATP2A3; TM4SF5; DNAH2; TMEM238L; TVP23C; LGALS9C; LGALS9B; TMEM11; MTRNR2L1; NOS2; TMEM97; VTN; RPL23A; PIPDX; UTP6; TMEM98; CCL15-CCL14; LHX1; TBC1D3L; SRCIN1; CWC25; PPP1R1B; GSDMB; IGFBP4; KRTAP9-4; KRT34; MYL4; AC003665.1; PRR15L; HOXB7; PRAC2; TTLL6; GIP; ITGA3; COL1A1; ABCC3; AKAP1; USP32; KCNH6; PRKCA; NOL11; PRKAR1A; KCNJ16; RAB37; HID1; CDR2L; MYO15B; ITGB4; TSPAN10; THOC1; PIEZO2; LAMA3; ZNF521; KCTD1; TTR; MAPRE2; RPL17-C18orf32; MEX3C; CCDC68; ONECUT2; ATP8B1; PMAIP1; TNFRSF11A; C18orf63; TPGS1; MISP; EFNA2; AMH; GNG7; AC006538.1; GNA11; SMIM24; LRG1; PTPRS; XAB2; CLEC4G; P2RY11; C19orf38; CNN1; AC022415.2; SYCE2; ADGRE5; ADGRE2; CASP14; CYP4F12; OR10H5; BST2; COLGALT1; MAST3; PGPEP1; LRRC25; TM6SF2; ZNF99; AC092329.3; ZNF302; FFAR2; CAPN12; LGALS4; CLC; FCGBP; RAB4B-EGLN2; LYPD4; CD79A; RABAC1; CD177; PLAUR; ZNF235; PVR; CBLC; NOVA2; GRIN2D; DBP; CGB7; TRPM4; FCGRT; RCN3; SIGLEC8; ZNF845; NCR1; TMEM86B; PEG3; ZNF543; CENPB; CDC25B; CHGB; PLCB1; PAK5; BTBD3; MACROD2; PYGB; TLDC2; FAM83D; HNF4A; ACOT8; ZSWIM3; PARD6B; BCAS1; PFDN4; CDH26; BTG3; TMPRSS15; TIAM1; B3GALT5; MX2; MX1; ABCG1; SIK1; COL6A1; C21orf58; PRMT2; ADA2; GGTLC3; RANBP1; RTN4R; TMEM191C; GGT1_1; PITPNB; TBC1D10A; GAL3ST1; SELENOM; APOL2; APOL1; CARD10; LGALS2; SLC16A8; KDELR3; APOBEC3B; PNPLA3; TTC38; IL17REL; TYMP; PLCXD1; ARSE; MXRA5; VCX3A; PIR; TXLNG; SH3KBP1; KLHL15; OTC; TIMP1; SSX5; PCSK1N; SYP; HEPH; TEX11; GJB1; AL133500.1; PBDC1; TCEAL7; RADX; MORC4; CLDN2; VSIG1; TMEM164; CAPN6; CT83; DAZ2; MT-CO1; and/or MT-CO2.

In certain aspects, the list of genes is selected from FosB, Early growth response protein 1 (EGR1), Early growth response protein 3 (EGR3), Nuclear receptor subfamily 4 group A member 1 (NR4A1), Cyclic AMP-dependent factor ATF-3 (ATF3), Hepatocyte nuclear factor 4-alpha (HNF4A), Ankyrin repeat and SAM domain-containing protein 4B (ANKS4B), Galectin-4 (LGALS4), Apoptosis facilitator Bcl-2-like protein 14, Cyclin-dependent kinase inhibitor 2A (CDKN2A), Matrix metalloproteinase 7 (MMP7), E3 ubiquitin-protein ligase Mdm2 (MDM2), Regenerating islet-derived protein (REG), Calpain 8 (CAPN8), Defensin beta 103A (DEFB103A), Rho GTPase Activating Protein 26 (ARHGAP26), Cell division cycle 25B (CDC25B), and G protein subunit beta-2 (GNB2).

The probiotic bacteria, prebiotic agents, and/or xenobiotics for use with the present invention can be provided in a variety of dosage forms. For example, e.g., tablets, capsules, pills, powders, granules, elixirs, tinctures, suspensions, syrups, enemas, suppositories, and emulsions may be used to provide the probiotic bacteria, prebiotic agents, and/or xenobiotics of the present invention to a patient in need of therapy for Barrett's esophagus.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference, and the like, relevant portions incorporated herein by reference.

As used herein, the term “treatment” refers to the treatment of the conditions mentioned herein, particularly in a patient who demonstrates symptoms of Barrett's esophagus. As used herein, the term “treating” refers to any administration of a compound of the present invention and includes (i) inhibiting the disease in an animal that is experiencing or displaying the pathology or symptomatology of the diseased (i.e., arresting further development of the pathology and/or symptomatology) or (ii) ameliorating the disease in an animal that is experiencing or displaying the pathology or symptomatology of the diseased (i.e., reversing the pathology and/or symptomatology). The term “controlling” includes preventing, treating, eradicating, ameliorating or otherwise reducing the severity of the condition being controlled.

As used herein, the terms “effective amount” or “therapeutically effective amount” refer to an amount of a subject compound or probiotics that will elicit the biological or medical response to Barrett's esophagus that is being sought by the researcher, veterinarian, medical doctor or other clinician.

As used herein, the terms “administration of” or “administering a” when referring to a compound should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as oral, esophageal, intranasal, and the like; inhalation powders, sprays, suspensions, and the like.

In this study, an ex vivo model for examining the mechanism by which host-microbe interactions occur within the esophagus is described. Using a monolayer of immortalized esophageal cells with an air-liquid interface, a normal microbiome and Barrett's microbiome will be transplanted directly onto the epithelial cell layer. This model can be manipulated to examine the mechanism by which different microbial communities interact with the esophageal mucosa and the differences in transcription and metabolomics profiles. Also described are experiments performed to characterize the microbiome and transcriptome profile of the human esophagus. This human data allows for a direct comparison with the results from the ex vivo experiments.

Esophageal Microbiome. Study participants. The first aspect of the experiment was to perform the esophageal microbiome analysis. After institutional review board approval was obtained, 74 patients were recruited into the study. Patient demographics are shown in Table 1. All participants were 1) patients undergoing surveillance endoscopy for a known history of Barrett's esophagus or 2) patients for whom screening endoscopy was recommended or could be considered based on guidelines from the American College of Gastroenterology. Indications for screening included men or women with chronic symptoms (greater than 5 years) of gastroesophageal reflux disease (GERD) and two or more risk factors for Barrett's esophagus or esophageal adenocarcinoma: Caucasian race, age≥50 years, chronic GERD symptoms, current or prior history of smoking, central obesity as defined as a waist circumference greater than 88 centimeters, waist to hip ratio greater than 0.8, family history of Barrett's esophagus or family history of esophageal adenocarcinoma [11]. GERD was defined as a condition in which the reflux of stomach contents into the esophagus led to symptoms of heartburn, regurgitation or other signs related to reflux.

Demographic data was not available for the first 5 patients in the Barrett's group. The average age in the entire cohort was 60.2 years. The majority of patients were currently on proton pump inhibitor therapy at the time of endoscopy, including 97% of the Barrett's group and 88% of the GERD without Barrett's group. Ten patients had long-segment Barrett's esophagus, defined as a length of Barrett's esophagus of three centimeters or greater.

TABLE 1
Esophageal Microbiome Cohort Demographics*.

| | Barrett's | GERD without | |
|---|---|---|---|
| | esophagus | Barrett's | p-value |
| N | 34 | 40 | |
| Male | 62% (18/29) | 50% (20/40) | 0.32 |
| Age, years (mean) | 61.7 ± 10.7 | 59.0 ± 8.9 | 0.26 |
| BMI (mean) | 31.5 ± 8.7 | 31.1 ± 5.5 | 0.83 |
| Hiatal hernia | 52% (15/29) | 35% (14/40) | 0.16 |
| Current smoker | 24% (7/29) | 20% (8/40) | 0.61 |
| Current PPI use | 97% (28/29) | 88% (35/40) | 0.23 |
| Mean PPI dose | 46.2 | 37.0 | 0.11 |
| (milligrams) | | | |

*Demographic data was unavailable in 5 patients in the Barrett's esophagus group.

When comparing the Barrett's group to the GERD without Barrett's group, there were no significant differences in age, gender ratio, BMI, tobacco use, presence of a hiatal hernia, current use of proton pump inhibitors or dose of proton pump inhibitors.

Clinical characteristics. Following endoscopy, physical examination and scripted interviews, the presence of Barrett's esophagus, age, gender, body mass index (BMI), ethnicity, presence of a hiatal hernia, smoking history and use/dose of proton pump inhibitors were recorded. For patients with Barrett's esophagus, the presence of dysplasia and the length of the Barrett's column were also recorded. Barrett's esophagus was defined as the presence of intestinal metaplasia in the distal esophagus. The presence of Barrett's esophagus was confirmed histologically in every patient included in this study.

Endoscopy. Prior to its use, the endoscope was sterilized and placed in a sterile container. The endoscope was then removed from the sterile container and placed directly into the esophagus. During the endoscopy, biopsies of the esophagus were taken from 1) normal esophagus from the proximal third of the esophagus (NP) and 2) normal esophagus from the distal esophagus, within one centimeter of the gastroesophageal junction (ND). In patients with Barrett's esophagus, a mucosal biopsy of normal esophagus was taken within one centimeter of the gastroesophageal junction and adjacent to the Barrett's esophagus. A swab of the uvula was also obtained using a sterile swab and immediately before the endoscopy was begun.

DNA extraction. Mucosal swabs and tissue biopsies were placed into sterile Powerbead tubes pre-loaded with 0.1 mm glass beads (Qiagen, Germantown, M D) plus external lysis buffer in vitro diagnostic (200 μL, Roche Applied Science, Indianapolis, Ind.). Tissues were homogenized at 30 Hz for 5 minutes using a Tissuelyser II homogenizer (Qiagen). Sample lysates were deposited into individual wells of 96 deep-well processing plates. DNA was subsequently extracted in high-throughput fashion using a MagNA Pure 96 instrument running a DNA and viral small volume-in vitro diagnostic extraction kit according to the manufacturer's protocol (Roche). After extraction, a portion of the DNA was evaluated by Ion Torrent Next Generation Sequencing or using the Esophageal Microbiome Array (EMB). The remaining material was archived at −20C.

Next Generation Sequencing. Sample sequencing was carried out using a fusion-PCR method. Briefly, fusion-primers were designed in accordance with the manufacturer's guidelines (Ion Amplification Library Preparation—Fusion Method, Life Technologies, Carlsbad, Calif.) using Ion Xpress Barcodes linked to 16S gene primer pairs targeting hyper-variable regions 1-8 [12]. Each 25 μl PCR was carried out using: 12.5 μl iQ supermix™ (Bio-Rad, Hercules, Calif.), 1 μl of both forward and reverse (5 μM) primers, 9.5 μl nuclease-free water and 1 μl of DNA template. A total of 3 biopools of DNA created by equimolar mixing of the first 5 patient samples were analyzed. Each biopool represented DNA from the uvula swab, the NP mucosal tissue or the ND mucosal tissue. The DNA biopools were then used as templates for creation of subsequent fusion 16S libraries. PCR was completed in a c1000 thermocycler (Bio-Rad) using the following parameters: Cycle 1), 95 C, 3 minutes, Cycle 2), Step 1: 95 C, 45 seconds; Step 2: Primer-specific annealing temps., 45 seconds; Step 3: 72 C, 2 minutes, repeat 39×; Step 4: 72 C, 7 minutes. PCR products were purified using Qiagen Qiaquick spin-columns and quantified using a spectrophotometer (Bio-Rad). PCR products were then diluted, mixed in equal proportion and sequenced on an Ion Torrent GeneStudio S5 System using Ion 520 sequencing kits together with 520 size chips following the manufacturer's instructions (Life Technologies).

Bioinformatics for Ion Torrent. After generation, sequencing reads were filtered for quality and binned according to Ion Xpress barcode using Ion Torrent Suite software version 5.10.0. Sequencing reads in FASTQ format were further processed using web-based Galaxy software [13]. First, raw FASTQ files were normalized using the FASTQ groomer tool function. Next, each barcoded read was trimmed to remove the primer sequence and subsequently filtered to the expected size of the 16S gene target. After this level of processing, the sequence reads were concurrently compared to the SILVA 16S database using bowtie 2 software [14-15]. This yielded a call to species or genera level as well as the number of times each sequence matched the database (hit-rate). When multiple calls to a genus were made, the number of hits were added accordingly. These numbers were then converted to percentage of total to give an overall ratio of the sequenced sample.

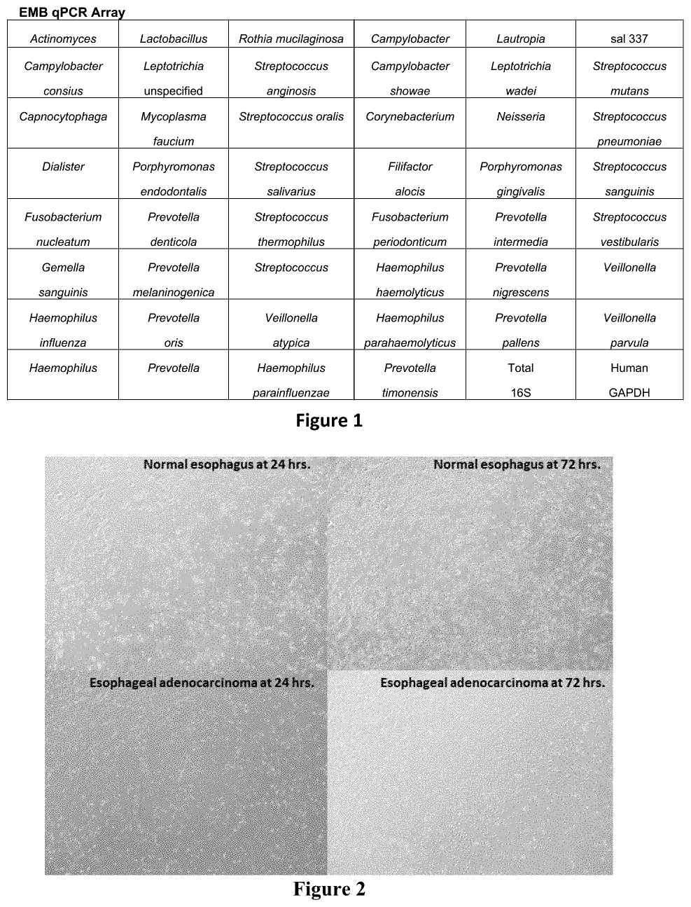

qPCR Evaluation by EMB. To construct the EMB, Ion Torrent data and information from the esophageal disease literature [16-23] were compiled to select the most commonly detected organisms from the uvula to the distal esophagus. Ultimately a list of 46 targets was created that collectively represented greater than 85 percent of the detected microbiota in the Ion Torrent sequencing datasets. Two control qPCR targets were added to address the human DNA (hGAPDH) and total bacterial genomic loads (total 16S), creating a 48-target panel that was constructed in a skirted 96-well plate format (ThermoFisher Scientific Inc.). The 48-target array was constructed in 6×8 format allowing for evaluation of 2 samples per 96 well plate (FIG. 1). Each 25 μl PCR was carried out using: 12.5 μl iQ SYBR green supermix™ (Bio-Rad), 1 μl of each forward and reverse (5-10 μM) primer, 9.5 μl nuclease-free water and 1 μl of DNA template. qPCR was completed in a c1000 thermocycler equipped with a CFX™ reaction module (Bio-Rad) using the following parameters: Cycle 1), 95 C, 3 minutes, Cycle 2), Step 1: 95 C, 30 seconds, Step 2: annealing 60 C, 30 seconds, extension 72 C, 30 seconds repeat 39×, Step 3: 72 C, 2 minutes, Step 4: Melt-curve 75 C-89 C, 0.2 C temperature increments with 5 second plate read time.

Fluorescent signal data was collected at the end of each annealing/extension step. Starting quantity values were extrapolated from standard curves of plasmids harboring the PCR targets previously confirmed by Sanger sequencing. Any organism that was below the limit of detection was categorized as not detected. Mathematical analyses were performed using Excel™ (Microsoft Corp., Redmond, Wash.).

Statistical analysis. The detection or non-detection of each organism was recorded in every sample in every patient. 2-way Firth-penalized logistic regression was used to relate the detection status to a selected variable (e.g., Barrett's esophagus vs. GERD without Barrett's esophagus) separately for each organism at each location. 2-way Firth-penalized logistic regression was used instead of conventional logistic regression due to the extreme values of detection incidence near 0% or 100% in many cases. The graphs for each organism were likewise modeled per 2-way Firth-penalized logistic regression, relating detection status to an association between a group (e.g., Barrett's esophagus vs. GERD without Barrett's esophagus) and a location (uvula, NP, ND). The graphs illustrate a model-predicted probability of detection at each location. To determine the association of the length of the Barrett's column with microbiota, Firth logistic regression was used for detection, restricted to the Barrett's esophagus group only, controlling for the covariates to determine the association between location and length of the Barrett's column. Statistical analyses were performed using R statistical software (R Core Team, 2018, version 3.5.1). In all statistical tests, α=0.05.

Transcriptome Profile. Study participants and sample collection. To evaluate the transcriptome profile, 37 patients with (n=9) and without (n=28) Barrett's esophagus were included in the analysis. Similar demographics were obtained and are shown in Table 2. Mucosal biopsies from the ND were used for the experiments.

TABLE 2
Esophageal Transcriptome Profile Cohort Demographics.

| | Barrett's esophagus | GERD without | p- |
|---|---|---|---|
| | group | Barrett's group | value |
| N | 9 | 28 | N/A |
| Age (years) | 63.3 | 61.2 | 0.58 |
| BMI | 26.9 | 31.5 | 0.03 |
| Hiatal hernia | 56% (5/9) | 32% (9/28) | 0.21 |
| Current smoker | 22% (2/9) | 25% (7/28) | 0.99 |
| Current PPI use | 89% (8/9) | 86% (24/28) | 0.81 |
| Mean PPI dose | 37.8 | 36.4 | 0.87 |
| (milligrams) | | | |

RNA Isolation. RNA was extracted using the Qiagen RNAeasy Micro Kit (Qiagen, Hilden, Germany). Esophageal mucosal biopsies were collected and stored in RNAeasy RLT lysis buffer with 1% B-mercaptoethanol. Samples were homogenized using a Bead mill 24 (Thermo Fisher Scientific, Pittsburgh, Pa.) at a speed of 5 meters/second for 30 seconds. Lysate slurry was centrifugated down and supernatant was collected for subsequent steps. 70% ethanol was added to the lysate and was transferred to an RNAeasy spin column. Spin column was washed using Qiagen RW1 buffer, RPE buffer and 80% ethanol. Final RNA collection was eluted with RNAse-free water. Final concentration was determined using a Qubit Fluorometer (Invitrogen, Carlsbad, Calif.).

Transcriptome Analysis. Total RNA was isolated from the mucosal biopsies and the RNA Integrity number was determined. Multiplexed RNA-seq libraries were generated for each sample using Smart-3SEQ protocol [24]. The libraries were sequenced on an Illumina NextSeq550 at an average depth of 4 million 75 base pair single end reads. The sequenced reads were trimmed and filtered based on adapter content and quality. The CLC Genomics Workbench 20.0 was used for the bioinformatical analysis of the RNA-Seq data. Filtered sequencing reads were locally alignment against the Homo sapiens (hg38) reference genome, with annotated genes and transcripts at minimum matching length and similarity fraction of 90%. Resulting gene counts were normalized, and differential expression analysis was performed on the complete list of genes and transcripts to evaluate the level and significance of mRNA expression changes [25]. All significantly altered genes underwent pathway analysis using Ingenuity Pathway Analysis to highlight molecular function, biological process and cellular component to assess the host's response. Clinical metadata, transcriptome profiles and microbial abundance were analyzed to identify significant associations between features from multiple measurement types. Hierarchical all-against-all association testing for correlation among all pairs of variables was performed using HAIIA 0.8.17. The resulting associations will be visualized in a network using Cytoscape 3.8.0 [26].

EX VIVO MODEL. Epithelial Cell Culture. Primary and immortalized cell lines have been created previously by the inventor using nasal and vaginal mucosa [27-29]. To create esophageal cell lines, immortalized cell lines were derived from human esophageal mucosal biopsies transformed with human papilloma virus E6E7. Primary and immortalized cell were cultured to form a monolayer of squamous cells representative of the esophageal epithelium. Normal esophagus and adenocarcinoma immortalized cell lines are shown at both 24 hours and 72 hours (FIG. 2).

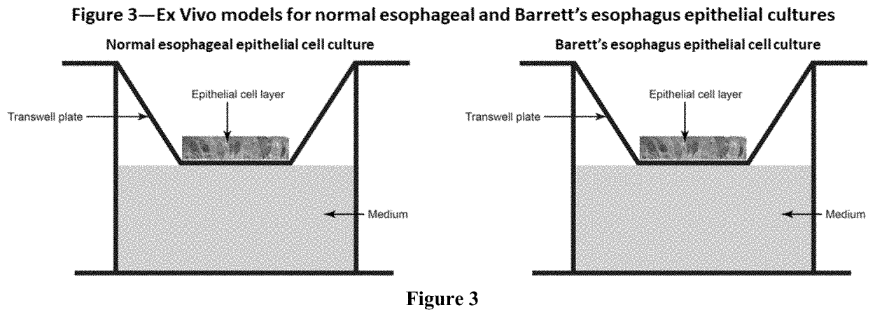

The monolayer cultures were cultivated in antibiotic-free keratinocyte serum-free medium (KSFM; Invitrogen, Carlsbad, Calif., USA) with 50 micrograms/milliliter (ug/ml) bovine pituitary extract, 50 ug/ml bovine pituitary extract, 44.5 ug/ml calcium chloride, and 0.2 mg/ml primocin (InvivoGen, San Diego, Calif., USA). A monolayer model was created in the same fashion using epithelial cells of diseased Barrett's esophagus. The monolayer was grown in a 33.6 square millimeter transwell cup (Fisher Scientific, Waltham, Mass., USA). The ex vivo models are shown in FIG. 3. The ex vivo model is ideal for esophageal microbiome testing for several reasons: 1) The apical surface of the epithelial squamous cell layer is exposed to air as in the esophagus, 2) The basal surface of the epithelial layer is in contact with a nutrient-rich medium and 3) the apical surface is sterile and can have various microbial communities transplanted for causative testing. It is important that the basal surface only is exposed to the nutrient-rich medium. This design of the model will allow the transplanted microbiomes to find nutrients in a similar fashion as in the human esophagus. This novel study design has allowed us to create experiments investigating potential mechanisms by which the esophageal microbiome causes disease. Though the ex vivo system is not an exact mimic, it does provide a good model for these studies.

Microbiome transplant. To prepare the microbiome transplant, mucosal biopsies of patients with and without Barrett's esophagus have been collected. Within 30 minutes of collection, the microbiota were harvested from the samples and stored in lysate. Multiple normal samples were pooled to create the “normal microbiome,” and multiple Barrett's samples were pooled to create the “Barrett's microbiome.” Sample pooling yield an equivalent normal microbiome and Barrett's microbiome. In addition, the harvesting and processing of samples is identical to experiments which measured the normal and Barrett's microbiomes. The lysate from each microbiome will be inoculated directly onto the epithelial layer of normal and Barrett's EC cultures.

The inventor's laboratory has previously performed this microbiome transplantation of the nasal microbiome. In that previous study, 20 different microbiome samples were transplanted onto nasal ex vivo models. The majority of the transplanted microbial communities mimicked the pre-transplant in vivo communities [27].

Esophageal Microbiome. Demographics. A total of 74 total patients were enrolled in the study, including 34 patients in the Barrett's group and 40 patients in the GERD without Barrett's group.

Microbiota detection patterns in patients with and without Barrett's esophagus. The detection or non-detection of each organism was recorded in every sample in every patient. 2-way Firth-penalized logistic regression was used to relate the detection status to a selected variable (e.g. Barrett's esophagus vs. GERD without Barrett's esophagus) separately for each organism at each location. 2-way Firth-penalized logistic regression was used instead of conventional logistic regression due to the extreme values of detection incidence near 0% or 100% in many cases. The graphs for each organism were likewise modeled per 2-way Firth-penalized logistic regression, relating detection status to an association between a group (e.g. Barrett's esophagus vs. GERD without Barrett's esophagus) and a location (uvula, NP, ND). The graphs illustrate a model-predicted probability of detection at each location. To determine the association of the length of the Barrett's column with microbiota, Firth logistic regression was used for detection, restricted to the Barrett's esophagus group only, controlling for the covariates to determine the association between location and length of the Barrett's column. Statistical analyses were performed using R statistical software (R Core Team, 2018, version 3.5.1). In all statistical tests, α=0.05.

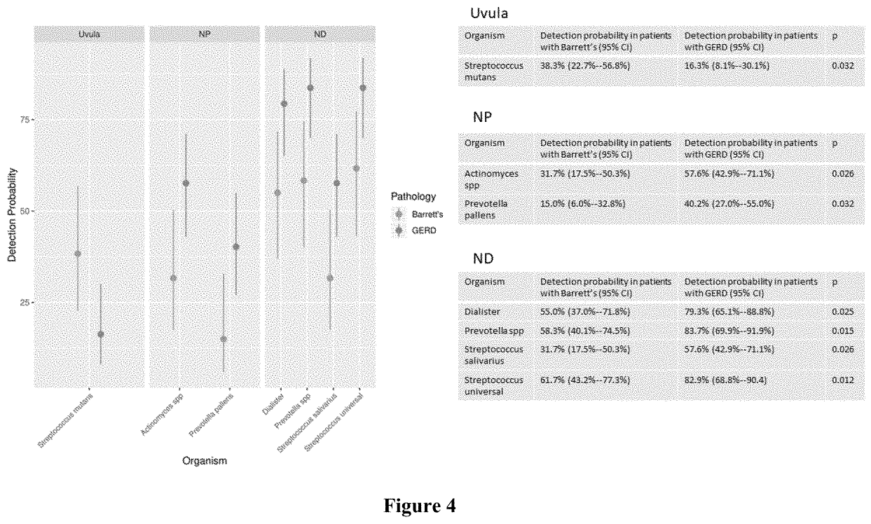

There were statistically significant differences in the likelihood of detection of multiple organisms in the Barrett's group compared to the GERD without Barrett's group. There were significant differences in likelihood of detection in one species (Streptococcus mutans) at the uvula, two genera or species (Actinomyces, Prevotella pallens) at the NP and four genera or species (Dialister, Prevotella unspecified, Streptococcus salivarius, Streptococcus unspecified) at the ND (FIG. 4).

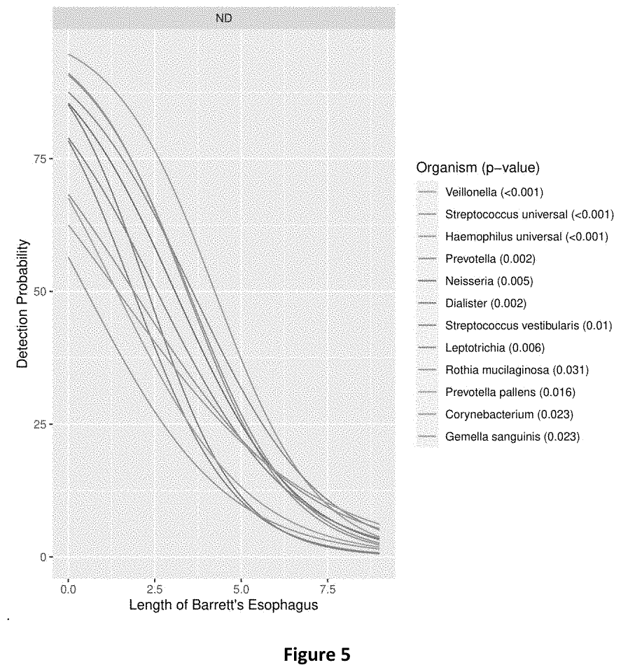

Severity of Barrett's Esophagus Versus Microbiome Pattern. There was a decreased likelihood of detection of multiple organisms as the length of the Barrett's column increased. In particular, 10 different genera (Corynebacterium, Dialister, Gemella, Haemophilus, Leptotrichia, Neisseria, Prevotella, Rothia, Streptococcus, Veillonella) on the EMB array had a significantly decreased likelihood of detection as the length of the Barrett's column increased (FIG. 5). Given that 2-way Firth-penalized logistic regression was used, the curves show the probability of detection of an organism being detected for any length of Barrett's esophagus. The only curves included in FIG. 5 are for organisms for which length is a significant predictor at an α≤0.05.

Transcriptome Profile. Demographics. Patient demographics are shown in Table 2. Average age was 61.7 years. Barrett's esophagus was present in 24% (9/37) of patients. Average BMI was 30.4. No patients with Barrett's esophagus had dysplasia. When analyzing patients with and without Barrett's esophagus, the only demographic which was significantly different between the two groups was BMI.

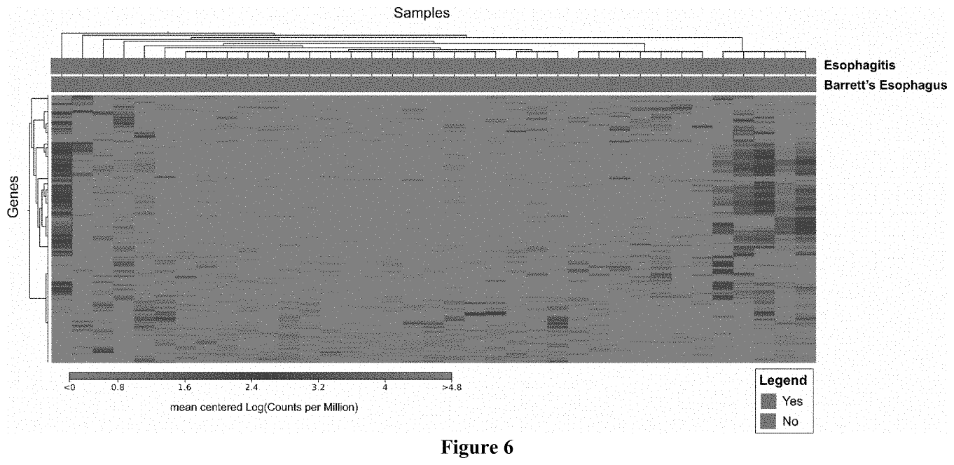

Cluster analysis. There was a distinct transcriptome profile seen in patients with Barrett's esophagus. Cluster analysis data is shown in FIG. 6. There were a large number of genes which were differentially expressed when comparing the two groups.

Many of these differentially expressed genes are involved in cell motility, apoptosis, stress response and biological processes typified in highly replicative malignant tissues. A partial number of these genes is listed in Table 3. For a complete list, see the supplementary table containing all differentially expressed genes.

TABLE 3
Partial list of genes differentially expressed in
Barrett's esophagus compared to normal.

| Transcription factor | Function |
|---|---|
| FosB | DNA binding |
| Early growth response protein 1 (EGR1) | Tumor suppressor |
| Early growth response protein 3 (EGR3) | Tumor suppressor |
| Nuclear receptor subfamily 4 group A | Energy homeostasis |
| member 1 (NR4A1) | |
| Cyclic AMP-dependent factor ATF-3 (ATF3) | Transcription repressor |
| Hepatocyte nuclear factor 4-alpha (HNF4A) | Transcription repressor |
| Ankyrin repeat and SAM domain-containing | Epithelial brush |
| protein 4B (ANKS4B) | differentiation |
| Galectin-4 (LGALS4) | Cell assembly |
| Apoptosis facilitator Bcl-2-like protein 14 | BCL2L14 |
| Cyclin-dependent kinase inhibitor 2A (CDKN2A) | Cell proliferation |
| Matrix metalloproteinase 7 (MMP7) | Cell degradation |
| E3 ubiquitin-protein ligase Mdm2 (MDM2) | Apoptosis |
| Regenerating islet-derived protein (REG) | Inflammation |
| Calpain 8 (CAPN8) | Cellular apoptosis |
| Defensin beta 103A (DEFB103A) | Regulation of cytokine |
| | production |
| Rho GTPase Activating Protein 26 (ARHGAP26) | Protein coding |
| Cell division cycle 25B (CDC25B) | Regulation of mitosis |
| G protein subunit beta-2 (GNB2) | Antiproliferative |
| | function |

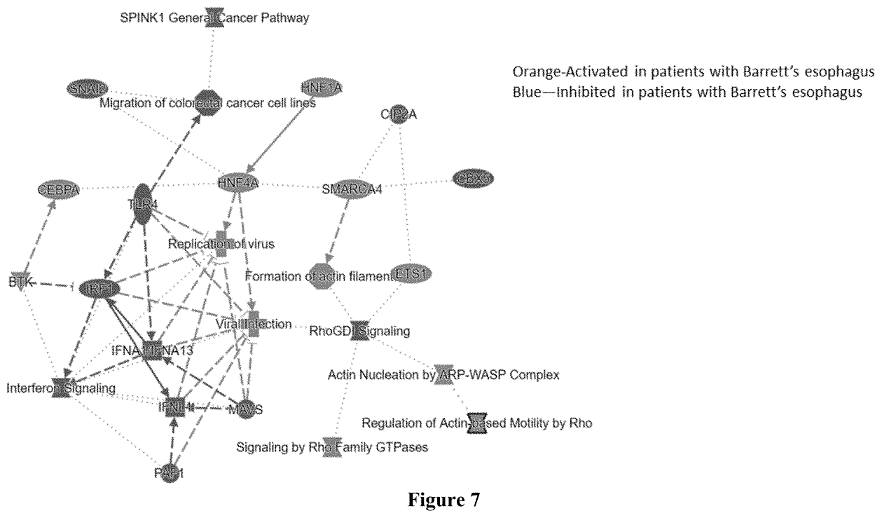

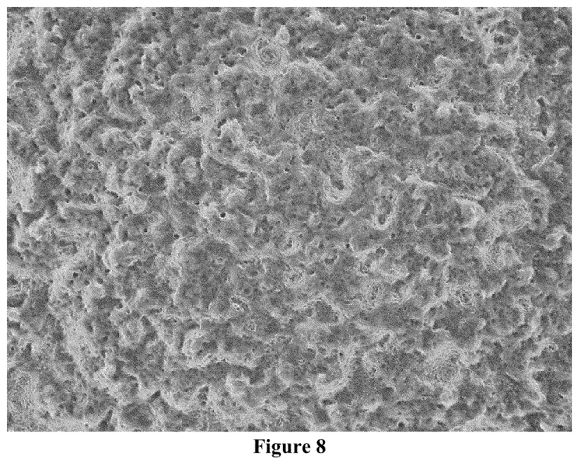

Ingenuity Pathway Analysis. Ingenuity Pathway Analysis was used to map specific functions and biological processes which were related to differences in transcriptome profile (FIG. 7).

EX VIVO MODEL. Epithelial cell layer. An epithelial cell layer of squamous cells was successfully grown onto the ex vivo model (FIG. 8). The microbiome transplants will be inoculated onto the apical surface of the cells. The resultant microbiome will be measured.

Transcriptome analysis after transplant. The supernatant in the ex vivo model will be collected. The resultant transcriptome profile will be measured.

There is a dynamic relationship between a host and its surrounding microbial community. A disruption in this symbiotic relationship can lead to adverse consequences. Other organ systems have seen a disturbance in the microbiome result in disease and worsening health status [30-32]. Helicobacter pylori levels have been associated with GERD and esophageal cancer in some studies, but this trend has been inconsistent [33-34]. Bacteria can act as direct promoters of cancer progression, but can also shield the host from disease [35]. In an effort to elucidate details of the complex role of the microbiome in esophageal disease, variations in the microbial community with esophageal disease were determined. An ex vivo model was created that allows for robust causative testing.

These results show that there are organisms in the esophagus which have a protective effect against esophageal disease. In these studies, a decreased frequency of detection of certain organisms in the presence of Barrett's esophagus was demonstrated. It was also showed that the detection of these organisms decreases as the length of Barrett's esophagus increases. Both of these clinical conditions are risk factors for esophageal cancer [36-38]. As such, it was postulate that similar bacterial trends may be associated with the development of esophageal cancer. These studies also show that there is a distinct transcription profile in patients with Barrett's esophagus, with a large number of genes which were differentially expressed. As described above, these genes regulate critical pathways of tumor biology, including cell motility, apoptosis and cellular stress responses.

There is a paucity of data in human experiments. Previous literature has used much more expensive and less efficient experiments on human tissue. This design is inefficient and may explain the relative lack of information on this important clinical dilemma. The benefit of an ex vivo model is that microbiome experiments can be performed repeatedly in a controlled environment. And this environment would not be subject to any inter-patient variability which likely exists in a large human cohort. Other ex vivo esophageal models have been created [39-40]. But no previous literature has used an ex vivo esophageal model for microbiome studies. The ex vivo model is a good replication of the human esophagus, even if not an exact duplication and allows for control of the environment in sterile conditions. These studies show that a sterile epithelial layer can be grown in an air-interfaced system. This design will allow for investigations on the relationship between mucosal cell function and its surrounding environment.

The pathway analyses yielded potential mechanisms by differentially expressed genes may affect the esophagus. As an example, there were multiple differentially expressed genes in this study which are involved in tumor suppression such as the EGR-related genes. This set of genes raises the possibility that inhibition of tumor suppression may be a mechanism by which the microbiome can increase the risk of esophageal cancer development. Future studies will be needed to examine specific genes/proteins, but these identified pathways may allow for treatment possibilities or diagnostic testing to identify patients at increased risk of cancer development.

With the design of the present invention, the inventor was be able to perform measurements on the resultant microbiome after manipulations on the ex vivo system. This system will allow us to understand whether the microbiome shifts as a result of the change in esophageal mucosa, versus the microbial shift causing the esophageal mucosa to change. This design allows for investigation of cellular and histologic changes, such as proliferation rate and apoptosis. And this design will permit investigations into molecular changes caused by shifts in the esophageal microbiome.

One limitation of the ex vivo model is the possibility that the environment may not be a perfect surrogate for the human esophagus. In cell culture, epithelia may behave differently when culture with stromal cells than when cultured in a monolayer [41]. This design has the added advantage that it is a multi-layered epithelial surface, but further studies are needed to determine how well this system mimics the esophagus. The majority of patients in this study used proton pump inhibitors. And a recent study by the present invention showed that there were no differences in the esophageal microbiome in patients who did and did not use proton pump inhibitor medications [42].

There is a distinct microbiome in patients with Barrett's esophagus. There is also a unique transcriptome profile in these patients, with a large number of genes which are differentially expressed. Ultimately, experiments using this ex vivo model could create new surveillance regimens and therapeutic interventions. Probiotic supplements containing protective organisms could be administered to prevent the development of Barrett's esophagus and esophageal cancer. Surveillance strategies, which currently use histologic changes primarily, could include microbiome shifts to guide treatment.

Table 4. Genes associated with Barrett's esophagus and esophageal cancer, according to protein nomenclature in Genecards or UniProt, relevant amino acid and nucleic acid sequence information incorporated herein by reference.

ISG15; PRXL2B; ESPN; ERRFI1; EFHD2; AGMAT; FBLIM1; PADI2; RCC2; AKR7L; PLA2G2A; LDLRAD2; HTR1D; CNR2; PAFAH2; SLC9A1; IFI6; DNAJC8; SESN2; RAB42; MEAF6; BMP8B; GUCA2B; GUCA2A; RIMKLA; SVBP; TMEM53; BEST4; TOE1; LURAP1; RAB3B; AL445685.3; USP1; GADD45A; PTGER3; TNNI3K; IFI44L; SAMD13; GBP3; BRDT; PALMD; SLC35A3; SLC30A7; GPSM2; KIAA1324; PSMA5; KCND3; RHOC; PTPN22; BCL2L15; AMPD1; REG4; NBPF20; NBPF10; FMO5; AC239811.1; MTMR11; SELENBP1; FLG; LCE3E; LCE3D; SPRR2F; PRR9; S100A7A; S100A7; AQP10; CD1E; IFI16; SLAMF7; ITLN1; FCGR2C; GPA33; F5; Clorf112; SELE; SUCO; AXDND1; LAMC2; RGS2; CRB1; LGR6; FMOD; ZBED6; GOLT1A; IL19; CR1; PROX1; RRP15; MARK1; CAPN8; TP53BP2; LEFTY1; RHOU; DISC1; GCSAML; LYPD8; CMPK2; ID2; HPCAL1; APOB; ATAD2B; NCOA1; KCNK3; TRIM54; RASGRP3; GALM; PLEKHH2; EPCAM; CNRIP1; ASPRV1; PAIP2B; ZNF638; DUSP11; ACTG2; LRRTM4; REG3G; REG3A; KIAA1211L; IL1R2; IL1RL1; FHL2; SULT1C2; ZC3H8; TFCP2L1; MYO7B; POTEI; PSMD14; GCA; NOSTRIN; METTLS; PDE1A; FSIP2; TFPI; C2orf88; CAVIN2; C2orf66; BOLL; PPIL3; CDK15; NRP2; SPAG16; TNS1; AAMP; VIL1; DES; SPEGNB; GMPPA; ALPG; SNORC; UGT1A9; MLPH; HES 6; GPR35; GHRL; TAMM41; TIMP4; RAF1; AC090004.1; BTD; KCNH8; LRRFIP2; VILL; VIPR1; SLC6A20; CCDC12; LAMB2; TCTA; AC104452.1; MST1R; SEMA3B; HYAL1; ACY1; GLYCTK; CHDH; APPL1; CEP97; CD47; CD96; HGD; STXBPSL; HCLS1; MCM2; RHO; NME9; SLC9A9; TM4SF4; TSC22D2; SUCNR1; PQLC2L; ARL14; GOLIM4; TNFSF10; FXR1; LAMP3; C3orf70; IGF2BP2; SST; TP63; IL1RAP; FAM43A; CEP19; MFSD10; WFS1; MRFAP1L1; FGFBP2; PROM1; LDB2; GBA3; OCIAD2; LRRC66; CENPC; STAP1; UGT2B11; SULT1B1; SULT1E1; SLC4A4; CXCL6; CXCL1; CXCL5; CXCL2; CXCL9; CXCL10; LIN54; ABCG2; ADH6; MTTP; DAPP1; SLC9B1; UGT8; KIAA1109; HSPA4L; ZNF330; HHIP; OTUD4; SMIM31; PALLD; NEK1; GALNT7; HPGD; DCTD; ING2; CASP3; SORBS2; LRRC14B; SLC9A3; SLC12A7; SLC6A19; OTULINL; RETREG1; SUB1; DNAJC21; IL7R; DAB2; OXCT1; FST; PPWD1; SREK1; CCDC125; IQGAP2; F2RL2; TBCA; MTX3; XRCC4; HAPLN1; ADGRV1; TRIM36; TICAM2; SEMA6A; SLC22A5; SMIM32; PFDN1; PCDHA1; RNF14; ARHGAP26; CDX1; NMUR2; GALNT10; FAXDC2; SGCD; MED7; RANBP17; AC139491.7; CDHR2; FGFR4; CLK4; ZNF354B; ZFP2; BTNL3; GMPR; NUP153; TPMT; MBOAT1; DCDCl₂; SLC17A4; HIST1H1D; TRIM31; TRIM15; DHX16; CDSN; LY6G5B; DXO; PPT2-EGFL8; GPSM3; MLN; RPS10-NUDT3; SPDEF; SCUBE3; CLPS; RAB44; KCNK16; AL096814.1; KLC4; MEP1A; MTO1; IMPG1; NT5E; SYNCRIP; SMPD2; LAMA4; ASF1A; NCOA7; C6orf58; BCLAF1; ARFGEF3; ECT2L; CCDC170; ZDHHC14; SOD2_1; SLC22A1; PHF10; ADAP1; LFNG; FSCN1; ICA1; TSPAN13; AGR2; IGF2BP3; HOXA9; HOXA10; HOXA13; CPVL; MRPL32; COAL; PURB; IGFBP3; EGFR; PHKG1; GUSB; ASL; DNAJC30; CCL26; FGL2; CD36; SEMA3C; CACNA2D1; SEMA3A; SEMA3D; KIAA1324L; CROT; SRI; CFAP69; GTPBP10; CDK6; GNGT1; ASB4; ARPC1B; ZCWPW1; GNB2; MUC3A; MUC17; MOGAT3; PLOD3; CLDN15; RASA4; POLR2J2; SLC26A3; PNPLA8; CFTR; CTTNBP2; FLNC; TMEM209; CPA2; CPA4; CPA1; KIAA1549; PRSS1; PRSS2; KEL; TMEM139; OR6B1; PDIA4; AOC1; WDR86; NOM1; KBTBD11; DEFA5; DEFB4B; DEFB103B; DEFB103A; DEFB4A; SOX7; GATA4; C8orf58; TRIM35; HMBOX1; DUSP4; BAG4; ZMAT4; PLAT; LY96; TPD52; PAG1; PMP2; FABP4; RUNX1T1; CDH17; AC012213.5; EMC2; MRPL13; ANXA13; FER1L6; SLC45A4; TSNARE1; GSDMD; PPP1R16A; ACER2; MOB3B; C9orf72; SPINK4; ANKRD18B; CA9; ZCCHC7; PRUNE2; KIF27; ROR2; BICD2; FBP1; AOPEP; FOXE1; HEMGN; SMC2; TAL2; EPB41L4B; C9orf43; TRAF1; NEK6; CEL; GPSM1; MAMDC4; ENTPD2; NRARP; UCN3; TUBAL3; PFKFB3; PROSER2; FAM171A1; MLLT10; OTUD1; BAMBI; SVIL; ZNF438; ZNF485; ZFAND4; MSMB; GPRIN2; ZNF488; PARG; A1CF; PRKG1; IPMK; PBLD; RUFY2; HKDC1; SLC29A3; DNAJC9; MYOZ1; SYNPO2L; KCNMA1; NUTM2B; IFIT2; IFIT3; IFIT1; PLCE1; CYP2C19; CYP2C9; HOGA1; SEMA4G; LDB 1; NEURL1; SMC3; ADRA2A; VWA2; PNLIPRP1; PNLIPRP2; PRLHR; GRKS; CUZD1; GPR26; CLRN3; VENTX; PGGHG; IFITM3; CDHRS; CRACR2B; MUCSAC; SLC22A18AS; SLC22A18; PHLDA2; HBB; FAM160A2; CAVIN3; RIC3; STK33; SCUBE2; KCNJ11; ABCC8; PRMT3; GAS2; SVIP; SLC5A12; FIBIN; PAX6; TP53111; PTPRJ; OR8H1; SLC43A1; UBE2L6; SERPING1; MS4A2; MS4A1; PGA3; PGAS; FADS2; PLAAT3; PLCB3; BRMS1; LRFN4; C11orf86; AP003419.1; ACY3; TCIRG1; CHKA; C11orf24; ANAPC15; STARD10; PLEKHB1; KLHL35; PCF11; EED; RAB38; FUT4; MAML2; MMP1; POGLUT3; LAYN; TMPRSS5; HTR3B; APOA4; MPZL2; CD3E; CD3D; CD3G; CCDC153; USP2; GRAMD1B; BARX2; ADAMTS15; SLC6A12; DYRK4; C1S; C1R; APOBEC1; PHC1; CLEC2B; KLRK1; EPS8; BHLHE41; PTHLH; PUS7L; IRAK4; PCED1B; DNAJC22; GPD1; SMIM41; KRT74; KRT76; KRT8; KRT18; PRR13; HOXC11; HOXC10; MUCL1; METTL7B; SLC39A5; RBMS2; MYO1A; R3HDM2; METTL1; HMGA2; MYRFL; TSPAN8; TRHDE; NAV3; CCDC59; EEA1; TMCC3; SNRPF; SLC41A2; C12orf75; TMEM263; SSH1; TCTN1; PPP1CC; SH2B3; OAS2; TBX3; SRRM4; HNF1A; TMEM120B; RHOF; HIP1R; KMT5A; RFLNA; CDX2; VWA8; SLC25A30; HTR2A; MED4; LPAR6; AL162377.3; ATP7B; DACH1; GPR183; COL4A2; TEP1; RNASE2; TMEM253; OR5AU1; RABGGTA; LTB4R2; CTSG; SSTR1; PPM1A; ZBTB1; PPP1R36; PLEK2; TMEM229B; FAM161B; ENTPD5; FLVCR2; DIO2; RIN3; CHGA; ITPK1; IFI27L2; AMN; CKB; INF2; GPR132; IGHD; NPAP1; AC124312.1; CHRNA7; ZNF770; PHGR1; DISP2; KNSTRN; PPP1R14D; CHAC1; LTK; PLA2G4E; TGM5; GLDN; WDR72; RAB27A; PIGBOS1; DNAAF4; GCNT3; TLN2; TPM1; DAPK2; RBPMS2; SLC51B; CILP; SMAD6; C15orf61; CALML4; SEMA7A; CYP1A1; SCAPER; TSPAN3; STARD5; GOLGA6L9; AC245033.1; TM6SF1; FANCI; ARPIN-AP3S2; MEF2A; PDIA2; MCRIP2; MSLN; TPSB2; TPSAB1; C16orf91; GFER; SLC9A3R2; PRSS33; IL32; MTRNR2L4; SMIM22; NAGPA; C16orf89; GRIN2A; TEKTS; CPPED1; BFAR; MYH11; GP2; ANKS4B; AC092338.1; ERN2; CLN3; SULT1A2; SULT1A1; NPIPB12; ZG16; MAZ; PRRT2; AC009133.6; KCTD13; AC093512.2; SLX1A; ZNF423; HEATR3; BRD7; CHD9; RPGRIP1L; MT2A; MT1E; MT1H; CCL22; MMP15; CA7; CES3; TPPP3; HSD11B2; PARD6A; NRN1L; PRMT7; SMPD3; COG8; CLEC18A; CLEC18C; AC010547.4; MARVELD3; HP; LDHD; ZFP1; CTRB2; CTRB1; TMEM170A; AC009163.2; CHSTS; HSD17B2; ATP2C2; ZFPM1; SPIRE2; TUBB3; PRDM7; DOC2B; RILP; ASPA; P2RX5-TAX1BP3; HASPIN; P2RX1; ATP2A3; TM4SF5; DNAH2; TMEM238L; TVP23C; LGALS9C; LGALS9B; TMEM11; MTRNR2L1; NOS2; TMEM97; VTN; RPL23A; PIPDX; UTP6; TMEM98; CCL15-CCL14; LHX1; TBC1D3L; SRCIN1; CWC25; PPP1R1B; GSDMB; IGFBP4; KRTAP9-4; KRT34; MYL4; AC003665.1; PRR15L; HOXB7; PRAC2; TTLL6; GIP; ITGA3; COL1A1; ABCC3; AKAP1; USP32; KCNH6; PRKCA; NOL11; PRKAR1A; KCNJ16; RAB37; HID1; CDR2L; MYO15B; ITGB4; TSPAN10; THOC1; PIEZO2; LAMA3; ZNF521; KCTD1; TTR; MAPRE2; RPL17-C18orf32; MEX3C; CCDC68; ONECUT2; ATP8B1; PMAIP1; TNFRSF11A; C18orf63; TPGS1; MISP; EFNA2; AMH; GNG7; AC006538.1; GNA11; SMIM24; LRG1; PTPRS; XAB2; CLEC4G; P2RY11; C19orf38; CNN1; AC022415.2; SYCE2; ADGRE5; ADGRE2; CASP14; CYP4F12; OR10H5; BST2; COLGALT1; MAST3; PGPEP1; LRRC25; TM6SF2; ZNF99; AC092329.3; ZNF302; FFAR2; CAPN12; LGALS4; CLC; FCGBP; RAB4B-EGLN2; LYPD4; CD79A; RABAC1; CD177; PLAUR; ZNF235; PVR; CBLC; NOVA2; GRIN2D; DBP; CGB7; TRPM4; FCGRT; RCN3; SIGLEC8; ZNF845; NCR1; TMEM86B; PEG3; ZNF543; CENPB; CDC25B; CHGB; PLCB1; PAK5; BTBD3; MACROD2; PYGB; TLDC2; FAM83D; HNF4A; ACOT8; ZSWIM3; PARD6B; BCAS1; PFDN4; CDH26; BTG3; TMPRSS15; TIAM1; B3GALT5; MX2; MX1; ABCG1; SIK1; COL6A1; C21orf58; PRMT2; ADA2; GGTLC3; RANBP1; RTN4R; TMEM191C; GGT1_1; PITPNB; TBC1D10A; GAL3ST1; SELENOM; APOL2; APOL1; CARD10; LGALS2; SLC16A8; KDELR3; APOBEC3B; PNPLA3; TTC38; IL17REL; TYMP; PLCXD1; ARSE; MXRA5; VCX3A; PIR; TXLNG; SH3KBP1; KLHL15; OTC; TIMP1; SSX5; PCSK1N; SYP; HEPH; TEX11; GJB1; AL133500.1; PBDC1; TCEAL7; RADX; MORC4; CLDN2; VSIG1; TMEM164; CAPN6; CT83; DAZ2; MT-CO1; and/or MT-CO2

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±0.1, 0.5, 1, 2, 3, 4, 5, 6, 7, 10, 12 or 15%, or as understood to be within a normal tolerance in the art, for example, within 2 standard deviations of the mean. Unless otherwise clear from the context, all numerical values provided herein are modified by the term about.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Rustgi A, El-Serag H. Esophageal Carcinoma. N Engl J Med 2014; 71:2499-2509. DOI: 10.1056/NEJMra 1314530.
Hur C, Miller M, Kong C Y, Dowling E, Nattinger K, Dunn M, Feuer E. Trends in Esophageal Adenocarcinoma Incidence and Mortality. Cancer 2013; 119:1149-1158.
Coleman H, Xie S H, Lagergren J. The Epidemiology of Esophageal Adenocarcinoma. Gastroenterology 2018; 154:390-405.
Hvid-Jensen F, Pedersen L, Drewes A M, Sorensen H T, Funch-Jensen P. Incidence of adenocarcinoma among patients with Barrett's esophagus. N Engl J Med 2011; 365:1375-83.
Okereke I C, Hamilton C, Reep G, Krill T, Booth A, Ghouri Y, et al. Microflora composition in the gastrointestinal tract in patients with Barrett's esophagus. J Thorac Dis 2019; 11:S1581-S1587.
Snider E J, Freedberg D E, Abrams J A. Potential role of the microbiome in Barrett's esophagus and esophageal adenocarcinoma. Dig Dis Sci 2016; 61:2217-2225.
Baba Y, Iwatsuki M, Yoshida N, Watanabe M, Baba H. Review of the gut microbiome and esophageal cancer: Pathogenesis and potential clinical implications. Ann Gastroenterol Surg 2017 June; 1(2): 99-104. PMID: 29863142.
Yang L., Francois F., and Pei Z.: Molecular pathways: pathogenesis and clinical implications of microbiome alteration in esophagitis and Barrett esophagus. Clin Cancer Res 2012; 18:2138-44.
Koike Y, Li B, Lee C, Cheng S, Miyake H, Welsh C, Hock A, Belik J, Zani A, Perro A. Gastric emptying is reduced in experimental NEC and correlates with the severity of intestinal damage. J Pediatr Surg 2017; 52:744-8.
Takakura K, Hasegawa K, Goto Y, Muramatsu I. Nitric oxide produced by inducible nitric oxide synthase delays gastric emptying in lipopolysaccharide-treated rats. Anesthesiology 1997; 87:652-7.
di Pietro M, Chan D, Fitzgerald R C, Wang K K. Screening for Barrett's esophagus. Gastroenterology 2015; 148:912-23.
Klindworth A, Pruesse E, Schweer T, Peplies J, Quast C, Horn M, et al. Evaluation of general 16S ribosomal RNA gene PCR primers for classical and next-generation sequencing-based diversity studies. Nucleic Acids Res 2013; 41:el.
Blankenburg D, Gordon A, Von Kuster G, Coraor N, Taylor J, Nekrutenko A, et al. Manipulation of FASTQ data with Galaxy. Bioinformatics 2010; 26:1783-85.
Yilmaz P, Parfrey L W, Yarza P, Gerken J, Pruesse E, Quast C, Schweer T, Peplies J, Ludwig W, Glockner F O. The SILVA and “All-species Living Tree Project (LTP)” taxonomic frameworks. Nucleic Acids Res 2014; 42:D643-8.
Langmead B, Salzberg S L. Fast gapped-read alignment with Bowtie 2. Nat Methods 2012; 9:357-9.
Yang L, Chaudhary N, Baghdadi J, Pei Z. Microbiome in reflux disorders and esophageal adenocarcinoma. Cancer J 2014; 20:207-210.
Kaakoush N, Morris M. The oesophageal microbiome: an unexplored link in obesity-associated oesophageal adenocarcinoma. FEMS Microbiol Ecol 2016; 92(10). pii: fiw161.doi:10.1093/femsec/fiw16.
Paull G, Yardley J. Gastric and esophageal Campylobacter pylori in patients with Barrett's esophagus. Gastroenterology 1988; 95:216-218.
Deshpande N, Riordan S, Casta√±o-Rodr√±guez N, Wilkins M R, Kaakoush N O. Signatures within the esophageal microbiome are associated with host genetics, age, and disease. Microbiome 2018; 6:227.
Blackett K L, Siddhi S S, Cleary S, Steed H, Miller M H, Macfarlane S, et al. Oesophageal bacterial biofilm changes in gastro-oesophageal reflux disease, Barrett's and oesophageal carcinoma: association or causality? Aliment Pharmacol Ther 2013; 37:1084-1092.
Pei Z, Bini E J, Yang L, Zhou M, Francois F, Blaser M J. Bacterial biota in the human distal esophagus. Proc Natl Acad Sci 2004; 101:4250-4255.
Okereke I C, Miller A, Hamilton C, Booth A L, Reep G L, Andersen C L, et al. Microbiota of the oropharynx and endoscope compared to the esophagus. Sci Rep 2019 Jul. 15; 9(1):10201. doi: 10.1038/541598-019-46747-y.
Huang C H, Yu X, Liao W. The expensive-tissue hypothesis in vertebrates: Gut microbiota effect, a review. Int J Mol Sci 2018; 19. doi: 10.3390/ijms19061792.
Foley J W, Zhu C, Jolivet P, Zhu S X, Lu P, Meaney M J, West R B. Gene expression profiling of single cells from archival tissue with laser-capture microdissection and Smart-3SEQ. Genome Res 2019; 29:1816-25.
Robinson M D, McCarthy D J, Smyth G K. EdgeR: a Bioconductor package for differential expression analysis of digital gene expression data. Bioinformatics. 2010; 26:139-40.
Shannon P, Markiel A, Ozier O, Baliga N, Wang J, Ramage D, Amin N, Schwikowski B, Ideker T. Cytoscape: a software environment for integrated models of biomolecular interaction networks. Genome Res 2003; 13:2498-504.
Charles D D, Fisher J, Hoskinson S M, Medina-Colorado A A, Shen Y C, Chaaban M R, Widen S G, Eaves-Pyles T D, Maxwell C A, Miller A L, Popov V L, Pyles R B. Development of a novel ex vivo nasal epithelial cell model supporting colonization with human nasal microbiota. Front Cell Infect Microbio 2019; 21; 9:165. doi: 10.3389/fcimb.2019.00165.
Amerson-Brown M H, Miller A L, Maxwell C A, White M M, Vincent K L, Bourne N, Pyles R B. Cultivated human vaginal microbiome communities impact Zika and herpes simplex virus replication in ex vivo vaginal mucosal cultures. Front Microbiol 2019; 9:3340. doi: 10.3389/fmicb.2018.03340.
Gentry M, Taormina J, Pyles R B, Yeager L, Kirtley M, Popov V L, Klimpel G, Eaves-Pyles T. Role of primary human alveolar epithelial cells in host defense against Francisella tularensis infection. Infect Immun 2007; 75(8):3969-78.
Lynch S V, Pedersen O. The human intestinal microbiome in health and disease. N Engl J Med 2016; 15:2369-79.
Young V B. The role of the microbiome in human health and disease: an introduction for clinicians. BMJ 2017; 356:j831. doi: 10.1136/bmj.j831.
Lee J T, Kim C M, Ramakrishnan V. Microbiome and disease in the upper airway. Curr Opin Allergy Clin Immunol 2019; 19:1-6.
Xavier J B, Young V B, Skufca J, Ginty F, Testerman T, Pearson A T, Macklin P, Mitchell A, Shmulevich I, Xie L, Caporaso J G, Crandall K A, Simone N L, Godoy-Vitorino F, Griffin T J, Whiteson K L, Gustafson H H, Slade D J, Schmidt™, Walther-Antonio M R S, Korem T, Webb-Robertson B M, Styczynski M P, Johnson W E, Jobin C, Ridlon J M, Koh A Y, Yu M, Kelly L, Wargo J A. The Cancer Microbiome: Distinguishing Direct and Indirect Effects Requires a Systemic View. Trends Cancer. 2020; 6:192-204.
Blaser M J, Atherton J C. Helicobacter pylori persistence: biology and disease. J Clin Invest 2004; 113:321-33.
Song S, Vuai M S, Zhong M. The role of bacteria in cancer therapy—enemies in the past, but allies at present. Infect Agent Cancer 2018; 15; 13:9. doi: 10.1186/s13027-018-0180-y.
Hayakawa Y, Chang W, Jin G, Wang T C. Gastrin and upper GI cancers. Curr Opin Pharmacol 2016; 31:31-7.
Elias P S, Castell D O. The role of acid suppression in Barrett's esophagus. Am J Med 2017; 130:525-9.
Souza R F. From reflux esophagitis to esophageal adenocarcinoma. Dig Dis 2016; 34:483-90.
Sato T, Stange D E, Ferrante M, Vries R G, Van Es J H, Van den Brink S, Van Houdt W J, Pronk A, Van Gorp J, Siersema P D, Clevers H. Long-term expansion of epithelial organoids from human colon, adenoma, adenocarcinoma, and Barrett's epithelium. Gastroenterology 2011; 141:1762-72.
Neves A A, Di Pietro M, O'Donovan M, Waterhouse D J, Bohndiek S E, Brindle K M, Fitzgerald R C. Endoscopy 2018; 50:618-25.
Herbst-Kralovetz M M, Pyles R B, Ratner A J, Sycuro L K, Mitchell C. New systems for studying intercellular interactions in bacterial vaginosis. J Infect Dis 2016; 214:S6-13.
Tasnim S, Miller A, Jupiter D, Hamilton C, Reep G, Krill T, Pyles R, Okereke I C. Effects of Proton Pump Inhibitor Use on the Esophageal Microbial Community. BMC Gastroenterol 2020 Sep. 23; 20(1):312. doi: 10.1186/s12876-020-01460-3.

What is claimed is:

1. A method of detecting and treating a patient suspected of having Barrett's esophagus comprising:
obtaining a biological sample from an esophagus of the patient;
determining a transcriptome in the biological sample by detecting a presence of one or more genes selected from the one or more of the genes in Table 4; wherein
if the patient has a transcriptome indicative of an increased risk of esophageal cancer treating the patient with at least one of: removing at least part of the esophagus, esophagectomy, probiotic therapy, chemically targeting elimination of bacteria indicative of an increased risk of esophageal cancer or increasing a frequency of endoscopic surveillance, or chemotherapy.

2. The method of claim 1, wherein the genes are selected from 1, 2, 3, 4, 5, 6, 7, 8, 9, 10, 11, 12, 13, 14, 15 or more of the genes selected from FosB, Early growth response protein 1 (EGR1), Early growth response protein 3 (EGR3), Nuclear receptor subfamily 4 group A member 1 (NR4A1), Cyclic AMP-dependent factor ATF-3 (ATF3), Hepatocyte nuclear factor 4-alpha (HNF4A), Ankyrin repeat and SAM domain-containing protein 4B (ANKS4B), Galectin-4 (LGALS4), Apoptosis facilitator Bcl-2-like protein 14, Cyclin-dependent kinase inhibitor 2A (CDKN2A), Matrix metalloproteinase 7 (MMP7), E3 ubiquitin-protein ligase Mdm2 (MDM2), Regenerating islet-derived protein (REG), Calpain 8 (CAPN8), Defensin beta 103A (DEFB103A), Rho GTPase Activating Protein 26 (ARHGAP26), Cell division cycle 25B (CDC25B), and G protein subunit beta-2 (GNB2).

3. The method of claim 1, further comprising determining a biome in the biological sample wherein an absence of Streptococcus salivarius, Actinomyces, Prevotella, or Dialister is indicative of an increased risk of esophageal cancer.

4. The method of claim 1, further comprising determining a biome in the biological sample wherein an absence of Corynebacterium, Dialister, Gemella, Haemophilus, Leptotrichia, Neisseria, Prevotella, Rothia, Streptococcus, Veillonella is indicative of worsened severity of Barrett's esophagus and an increased risk of esophageal cancer.

5. The method of claim 1, further comprising stratifying patients based on the presence of bacteria in at least one of the uvula, proximal esophagus, and distal esophagus.

6. The method of claim 1, wherein the biological sample is an esophageal swab and mucosal biopsies were obtained from at least one of the uvula, proximal esophagus, and distal esophagus.

7. The method of claim 1, wherein the expression of 2, 3, 4, 5, 6, 7, 8, 9, 10, 11 or 12 genes is indicative of Barrett's esophagus.

8. The method of claim 1, wherein the genes are on an array.

9. The method of claim 1, wherein the biological sample is obtained from the distal esophagus and a length of a Barrett's column correlated with a level of Barrett's esophagus leading to esophageal cancer.

10. The method of claim 1, wherein the biological sample is a breath sample, a saliva sample, or a biopsy of the esophagus.

11. The method of claim 1, wherein the presence of Barrett's esophagus is determined without the use of age, gender, or presence of hiatal hernia as a factor or factors.

12. An ex vivo system of an esophagus comprising:
a well comprising a cellular growth media;
a substrate comprising an apical surface and a nutrient-rich basal surface on which an air-liquid interface is formed at the media, wherein an epithelial layer is grown on the apical surface; and
wherein a squamous epithelial layer of esophageal cells or cell lines is grown on the apical surface of the ex vivo system.

13. The model of claim 12, further comprising a microbiome harvested from one or more patients with Barrett's esophagus and putting in contact with the esophageal cells or cell lines.

14. The model of claim 12, further comprising one or more Barrett's esophageal cell lines.

15. The model of claim 12, wherein a microbiome is inoculated onto a sterile apical surface of each esophageal cell line.

16. The model of claim 12, wherein a microbiome is be inoculated onto a sterile apical surface a Barrett's esophageal cell line.

17. A kit for an ex vivo system of an esophagus comprising:
a well comprising a cellular growth media;
a substrate comprising an apical surface and a nutrient-rich basal surface on which an air-liquid interface is formed at the media, wherein an epithelial layer is grown on the apical surface adapted for the growth of a squamous epithelial layer of esophageal cells or cell lines is grown on the apical surface of the ex vivo system; and
instructions for the growth for a squamous epithelial layer of esophageal cells or cell lines.

18. A method of testing one or more compounds, microbiomes, or combinations there for efficacy in treating esophageal diseases in an ex vivo model of an esophagus comprising:
providing a well comprising a cellular growth media;
placing a substrate comprising an apical surface and a nutrient-rich basal surface on which an air-liquid interface is formed at the media, wherein an epithelial layer is grown on the apical surface;
growing squamous epithelial layer of esophageal cells or cell lines is grown on the apical surface of the ex vivo system;
contacting the squamous epithelial layer of esophageal cells or cell lines with the one or more compounds, microbiomes, or combinations; and
detecting a change in the squamous epithelial layer of esophageal cells or cell lines.

19. A method of determining an extent of disease progression in a patient suspected of having Barrett's esophagus comprising:
obtaining a biological sample from one or more locations of an esophagus of the patient;
determining a transcriptome in the biological sample by detecting a presence of one or more genes selected from: FosB, Early growth response protein 1 (EGR1), Early growth response protein 3 (EGR3), Nuclear receptor subfamily 4 group A member 1 (NR4A1), Cyclic AMP-dependent factor ATF-3 (ATF3), Hepatocyte nuclear factor 4-alpha (HNF4A), Ankyrin repeat and SAM domain-containing protein 4B (ANKS4B), Galectin-4 (LGALS4), Apoptosis facilitator Bcl-2-like protein 14, Cyclin-dependent kinase inhibitor 2A (CDKN2A), Matrix metalloproteinase 7 (MMP7), E3 ubiquitin-protein ligase Mdm2 (MDM2), Regenerating islet-derived protein (REG), Calpain 8 (CAPN8), Defensin beta 103A (DEFB103A), Rho GTPase Activating Protein 26 (ARHGAP26), Cell division cycle 25B (CDC25B), and G protein subunit beta-2 (GNB2); and
matching an extent of Barrett's esophagus to disease progression by detecting the presence of the one or more genes of the transcriptome at the one or more locations of the esophagus that correlate with different levels of Barrett's esophagus disease progression; and
if the patient has a transcriptome that is indicative or early Barrett's esophagus disease then increasing a frequency of Barrett's esophagus disease surveillance from every 3 months to every three years in three-month increments; or
if the patient has a transcriptome indicative of advanced Barrett's esophagus treating the patient with at least one of: removing at least part of the esophagus, esophagectomy, probiotic therapy, chemotherapy, chemically targeting elimination of bacteria indicative of an increased risk of esophageal cancer or increased frequency of endoscopic surveillance.

20. The method of claim 19, further comprising stratifying patients based on a presence of bacteria in at least one of the uvula, proximal esophagus, and distal esophagus.

21. The method of claim 19, wherein the biological sample is an esophageal swab and mucosal biopsies were obtained from the uvula, proximal esophagus and distal esophagus.

22. The method of claim 19, wherein expression of 2, 3, 4, 5, 6, 7, 8, 9, 10, 11 or 12 genes is indicative of Barrett's esophagus.

23. The method of claim 19, wherein the biological sample is obtained from the distal esophagus and a length of a Barrett's column correlated with a level of Barrett's esophagus leading to esophageal cancer.

24. The method of claim 19, wherein the biological sample is a breath sample, a saliva sample, a biopsy of the esophagus, or a serum sample.

25. The method of claim 19, wherein a presence of Barrett's esophagus is determined without use of age, gender, or presence of hiatal hernia as a factor or factors.
